(19) United States
(12) Patent Application Publication
(10) Pub. No.: US 2024/0117091 A1
(43) Pub. Date: Apr. 11, 2024

(54) OPACITY CHANGE IN PRINTING RESINS

(71) Applicant: VOCO GmbH, Cuxhaven (DE)

(72) Inventors: Sebastian Lamping, Münster (DE); Manfred Thomas Plaumann, Cuxhaven (DE)

(21) Appl. No.: 18/467,472

(22) Filed: Sep. 14, 2023

(30) Foreign Application Priority Data
Sep. 15, 2022 (DE) 10 2022 123 586.6

(51) Int. Cl. C08F 220/28; B33Y 70/00; C08F 220/30; C08F 222/10

(52) U.S. Cl. CPC C08F 220/282; B33Y 70/00; C08F 220/281; C08F 220/301; C08F 222/103; C08F 222/105; C08F 222/1063

(57) ABSTRACT

The present disclosure relates to dental, free-radically polymerizable compositions, and to the use thereof in stereolithography and/or 3D printing, in particular for the 3D printing of dental shaped components, such as dental models, trays, try-ins or dentures. The dental, free-radically polymerizable compositions feature a change in opacity during the polymerization.

### CROSS REFERENCE TO RELATED APPLICATIONS

This application claims priority to German Patent Application No. 10 2022 123586.6, filed Sep. 15, 2022, which is hereby incorporated by reference in its entirety.

### FIELD OF THE DISCLOSURE

The present disclosure relates to a free-radically polymerizable composition, and to the use thereof in stereolithography and/or 3D printing, preferably for the 3D printing of dental shaped components, such as dental models, trays, try-ins or dentures.

### BACKGROUND OF THE DISCLOSURE

With the constant advances in digital dentistry, additive manufacturing processes have in the meantime become established both in dentist practices and in dental laboratories. Data on the individual tooth situation are often obtained with a 3D scanner and can be processed on a computer. On the basis of these data, 3D printing or stereolithography can then be used to print dental shaped components depending on the application, such as for example models, trays, dentures, splints, occlusal splints, orthodontic shaped components, IBTs, casts, drilling templates, crowns or bridges.

Additive manufacturing methods, especially stereolithographic methods for producing dental shaped bodies, are known from the prior art.

WO 97/29901 A1 specifies a method for producing a three-dimensional article from a hardenable liquid medium, where the article is built up layer-by-layer by each time applying a layer of liquid medium to a support and/or an already formed part of the article in a vessel containing liquid medium and subsequently hardening said layer.

WO 2013/153183 A2 describes composite resin compositions and methods for producing dental components by means of stereolithography. The document claims the use of a dental composition, comprising a polyreactive binder, two photopolymerization initiators having different absorption maxima and an absorber.

For some applications the printed dental shaped components must be opaque. For example, dental models and trays require a certain opacity in order to be able to work with them. Crowns, bridges, try-ins and dentures likewise require opacity, in order to reproduce the natural situation in the mouth.

Dental materials that have a certain opacity after curing are known from the prior art. In particular, filling materials need to mirror the natural opacity of the tooth in order to give an esthetic, toothlike impression. At the same time, they must be permeable to the polymerization light in order to cure as completely as possible.

WO 2016/026915 A1 describes a dental filling material that prior to curing is essentially transparent (for the wavelength used in the polymerization) and after curing is opaque and hence corresponds to the natural tooth color. This problem is solved via the selection of the refractive indices of monomer matrix and fillers. Prior to the polymerization, the refractive indices coincide with one another well, and the material appears transparent. As a result of the polymerization and the shrinkage that occurs in the process, the refractive index of the monomer matrix increases during the transition to the polymer. After the polymerization, the refractive indices of polymer matrix and filler no longer coincide with one another and the material appears opaque.

Although this method can be applied very well in the case of highly filled dental filling materials, it cannot be transferred to polymerizable compositions for 3D printing. In order to exhibit this effect, the fillers require a certain particle size. However, such fillers are no longer sufficiently sedimentation-stable in the low-viscosity 3D printing compositions. While smaller, nanoscale fillers are more sedimentation-stable, they do not necessarily exhibit the same effects. Since the particle size lies far below the wavelength of visible light, such materials often appear transparent even after curing. On the other hand, such nanoscale fillers are complex to produce and are accordingly expensive.

There is therefore a need for simple, cost-effective compositions which prior to curing are transparent and hence readily permeable to the polymerization light and which after curing exhibit sufficient opacity for the dental application.

### BRIEF DESCRIPTION OF THE DISCLOSURE

In one aspect, the present disclosure is directed to a free-radically polymerizable composition comprising
A) 10% to 80% by weight of polymerizable (meth)acrylic compounds of formula I

Y—O—[(C₃H₆O)ᵥ-L¹]ₙ—(C₃H₆O)_{w}—Z (I)
wherein
Y=H₂C=C(R¹)—C(═O)— or H₂C=C(R¹)—C(═O)—X—L²-,
R¹=H or CH₃,
X=O or NH,
L¹=divalent organic linker group,
L²=divalent organic linker group,
Z=Y, H or C₁ to C₄ alkyl,
v=36 to 100,
w=36 to 200,
v+w=75 to 200,
n=0 to 3,
B) 15% to 85% by weight of polymerizable (meth)acrylic compounds of formula II

{H₂C=C(R¹)—C(═O)—X—[CH₂CH(R²)O]ₘ}_{q}Q (II)
wherein
R¹=H or CH₃,
R²=H or CH₃,
X=O or NH,
Q=a q-valent organic radical,
m=1 to 20,
q=1 to 4, and
C) 0.01% to 5% by weight of an initiator or initiator system for the free-radical polymerization,
based in each case on the total mass of the polymerizable composition.

In another aspect, the disclosure is directed to use of a free-radically polymerizable composition as described herein, for in stereolithography and/or 3D printing, preferably for the 3D printing of dental shaped components, with preference for the 3D printing of dental models, trays, try-ins or dentures.

In one particular aspect, the free-radically polymerizable compositions of the present disclosure are dental free-radically polymerizable compositions.

### DETAILED DESCRIPTION OF THE DISCLOSURE

In one aspect, the present disclosure is directed to a free-radically polymerizable composition comprising
A) 10% to 80% by weight of polymerizable (meth)acrylic compounds of formula I

Y—O—[(C₃H₆O)ᵥ—L¹]ₙ—(C₃H₆O)_{w}—Z (I)

wherein
Y=H₂C=C(R¹)—C(═O)— or H₂C=C(R¹)—C(═O)—X—L²_
R¹=H or CH₃,
X=O or NH,
L¹=divalent organic linker group,
L²=divalent organic linker group,
Z=Y, H or C1 to C4 alkyl,
v=36 to 100,
w=36 to 200,
v+w=75 to 200, and
n=0 to 3,
B) 15% to 85% by weight of polymerizable (meth)acrylic compounds of formula II

{H₂C=C(R¹)—C(═O)—X—[CH₂CH(R²)O]ₘ}_{q}Q (II)
wherein
R¹=H or CH₃,
R²=H or CH₃,
X=O or NH,
Q=a q-valent organic radical,
m=1 to 20,
q=1 to 4, and
C) 0.01% to 5% by weight of an initiator or initiator system for the free-radical polymerization,
based in each case on the total mass of the polymerizable composition.

In a preferred aspect, the free-radically polymerizable composition of the present disclosure is a dental free-radically polymerizable composition.

(Meth)acrylic compounds are understood here to mean both acrylic compounds and methacrylic compounds.

The use of monomers comprising polypropylene groups in 3D printing is known per se. For instance, WO 2019/175716 A1 describes a plurality of corresponding monomers. However, the monomers mentioned therein do not exhibit the desired effect. It is probable that the PPG units are not large enough.

WO 2022/097667 A1 also describes light-curable resin compositions comprising polyalkylene glycol monomers. The compositions are used for production of models in casting methods and feature low development of soot, which is intended to result in a reduction in cracks. A change in opacity is not described. Longer-chain monomers are disclosed only in connection with PEG units.

Surprisingly, it has been found that the combination of a (meth)acrylic compound of formula (I), which has one or more large PPG groups, and a (meth)acrylic compound of formula (II), which has one or more ethylene oxide or propylene oxide groups, results in compositions that are transparent prior to the polymerization and opaque after the polymerization.

“Transparent” is understood here to mean that the liquid monomer mixture is essentially permeable to light in the visible wavelength region. This applies in particular to the application-relevant layer thicknesses (for example the filling level of the resin vat of a 3D printer). The translucency or the corresponding opacity can be measured using a spectrophotometer as described hereinbelow. Essentially permeable means here that the translucency under the specified measurement conditions is ≥90%, preferably ≥95%, particularly preferably ≥97%, and very particularly preferably ≥99%. Accordingly, the opacity under the specified measurement conditions is <10%, preferably <5%, particularly preferably <3%, and very particularly preferably <1%.

Opaque is understood here to mean that the solid, polymerized test specimens are predominantly impermeable to light in the visible wavelength region. This applies in particular to the application-relevant layer thicknesses (for example the layer thickness of a crown or of a tray). The opacity can be measured using a spectrophotometer as described hereinbelow. Predominantly impermeable means here that the opacity under the specified measurement conditions is >50%, preferably >60%, and particularly preferably >70%.

A further advantage of the compositions according to the disclosure is that polymeric fragments, as for example can arise as a result of breaking off from the build platform, due to their opacity can be readily identified in the transparent resin and removed, whereas transparent fragments in transparent resins or opaque fragments in opaque resins can easily be overlooked.

### Polymerizable (Meth)Acrylic Compound (A)

In one aspect, a composition according to the disclosure comprises 10% to 80% by weight, preferably 10% to 70% by weight, particularly preferably 10% to 60% by weight, very particularly preferably 10% to 50% by weight, of polymerizable (meth)acrylic compounds of formula I

Y—O—[(C₃H₆O)ᵥ-L¹]ₙ—(C₃H₆O)_{w}—Z (I)

wherein
Y=H₂C=C(R¹)—C(═O)— or H₂C=C(R¹)—C(═O)—X—L²_
R¹=H or CH₃,
X=O or NH,
L¹=divalent organic linker group,
L²=divalent organic linker group,
Z=Y, H or C₁ to C₄ alkyl,
v=36 to 100,
w=36 to 200,
v+w=75 to 200, and
n=0 to 3.

The groups (C₃H₆O)ᵥ and (C₃H₆O)_{w} here are polypropylene glycol groups (PPG groups).

In a preferred embodiment, in the (meth)acrylic compound of formula I X=O, Z=Y, n=0 and w=75 to 180, preferably 75 to 150, particularly preferably 100 to 150.

In a further preferred embodiment, in the (meth)acrylic compound of formula I X=O, Z=Y, n=1 to 3, v=36 to 100, preferably 36 to 90, particularly preferably 36 to 80, w=36 to 100, preferably 36 to 90, particularly preferably 36 to 80, and v+w=75 to 180, preferably 75 to 150, particularly preferably 100 to 150.

In a further preferred embodiment, in the (meth)acrylic compound of formula I X=O, Z=Y, n=0 to 3, v=36 to 100, preferably 36 to 90, particularly preferably 36 to 80, w=36 to 100, preferably 36 to 90, particularly preferably 36 to 80, and n*v+w=75 to 200, preferably 75 to 180, particularly preferably 100 to 150.

In a preferred embodiment, in the (meth)acrylic compound of formula I, L¹ is a straight-chain, branched and/or cyclic alkylene, arylene, arylalkylene or alkylarylene group, which can contain —O—, —OC(═O)—, —C(═O)O—, —NHC(═O)—, —C(═O)NH—, —OC(═O)NH—, or —NHC(═O)O—, and L² is a straight-chain, branched and/or cyclic alkylene, arylene, arylalkylene or alkylarylene group, which can contain —O—, —OC(═O)—, —C(═O)O—, —NHC(═O)—, —C(═O)NH—, —OC(═O)NH—, or —NHC(═O)O—. Preferably, L¹ and/or L² is/are selected from the group consisting of

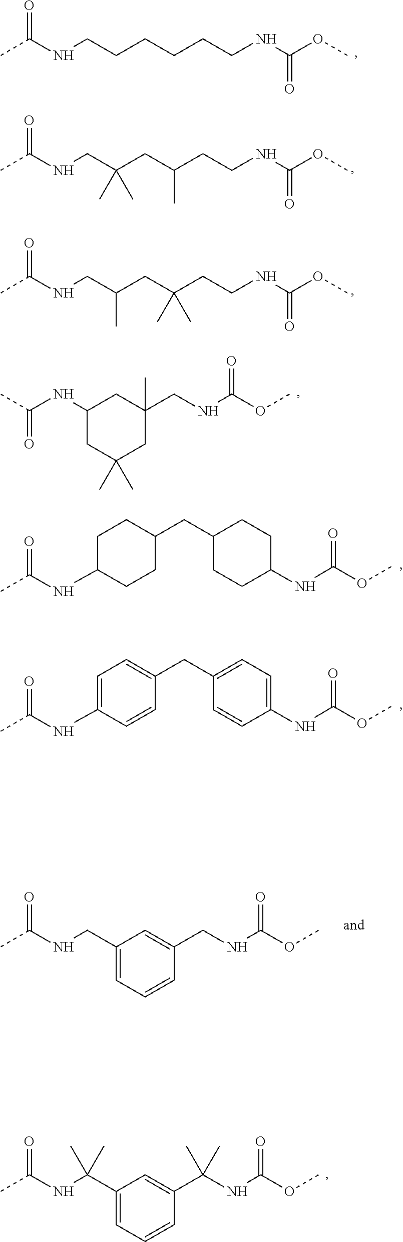

and preferably selected from the group consisting of

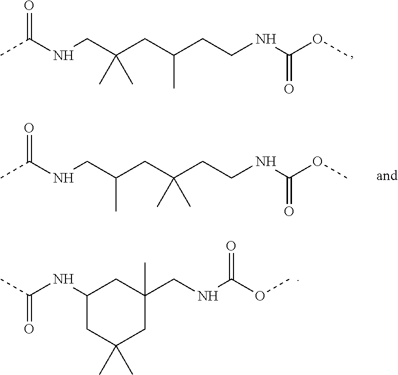

In some aspects, L¹ is bonded by the left-hand dashed line to the outer oxygen atom of the polypropylene group (C₃H₆O)ᵥ and is bonded by the right-hand dashed line to the outer carbon atom of the polypropylene group (C₃H₆O)_{w}, and/or L² is bonded by the left-hand dashed line to X and is bonded by the right-hand dashed line, via an oxygen atom (—O—), to the outer carbon atom of one of the polypropylene groups (C₃H₆O)_{w} or (C₃H₆O)ᵥ.

In a preferred embodiment, the (meth)acrylic compound of formula I has a molecular weight of greater than 5000 g/mol, preferably of greater than 6000 g/mol, particularly preferably of greater than 7000 g/mol, and/or in the range from 5000 to 15 000 g/mol, preferably in the range from 6000 to 12,000 g/mol, particularly preferably in the range from 7000 to 10,000 g/mol.

### Polymerizable (Meth)Acrylic Compound (B)

In one aspect, a composition according to the disclosure comprises 15% to 85% by weight, preferably 20% to 85% by weight, particularly preferably 30% to 85% by weight, very particularly preferably 40% to 85% by weight, of polymerizable (meth)acrylic compounds of formula II

{H₂C=C(R¹)—C(═O)—X—[CH₂CH(R²)O]ₘ}_{q}Q (II)

wherein
R¹=H or CH₃,
R²=H or CH₃, preferably H,
X=O or NH, preferably O,
Q=a q-valent organic radical,
m=1 to 20, and
q=1 to 4.

Preferably, X=O and/or R²=H.

In a preferred embodiment,

Q=H, C₁ to C₄ alkyl or phenyl, when q=1, or

Q=—(CH₂CH₂)—, —(CH₂CH(CH₃))— or

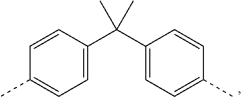

when q=2, or

Q=

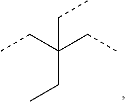

when q=3, or

Q=

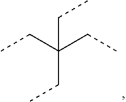

when q=4.

Preferred polymerizable (meth)acrylic compounds of formula (II) with q=1 are 2-hydroxyethyl (meth)acrylate, 2-hydroxypropyl (meth)acrylate, 2-(2-hydroxyethoxy)ethyl (meth)acrylate, 2-[2-(2-hydroxyethoxy)ethoxy]ethyl (meth)acrylate, ethoxylated 2-hydroxyethyl (meth)acrylate, 2-phenoxyethyl (meth)acrylate, 2-(2-phenoxyethoxy)ethyl (meth)acrylate, 2-[2-(2-phenoxyethoxy)ethoxy]ethyl (meth)acrylate and ethoxylated 2-phenoxyethyl (meth)acrylate.

Preferred polymerizable (meth)acrylic compounds of formula (II) with q=2 are ethylene glycol di(meth)acrylate, diethylene glycol di(meth)acrylate, triethylene glycol di(meth)acrylate, tetraethylene glycol di(meth)acrylate, polyethylene glycol di(meth)acrylate, propylene glycol di(meth)acrylate, dipropylene glycol di(meth)acrylate, tripropylene glycol di(meth)acrylate, tetrapropylene glycol di(meth)acrylate, polypropylene glycol di(meth)acrylate, ethoxylated bisphenol A di(meth)acrylate and propoxylated bisphenol A di(meth)acrylate.

Preferred polymerizable (meth)acrylic compounds of formula (II) with q=3 are ethoxylated trimethylolpropane tri(meth)acrylate and propoxylated trimethylolpropane tri(meth)acrylate.

Preferred polymerizable (meth)acrylic compounds of formula (II) with q=4 are ethoxylated pentaerythritol tetra(meth)acrylate and propoxylated pentaerythritol tetra(meth)acrylate.

In a preferred embodiment, the alkylene oxide units —[CH₂CH(R²)O]ₘ— make up at least 30% by weight of the molecular weight of the (meth)acrylic compound of formula II.

In addition to the (meth)acrylic compounds (I) and (II), a composition according to the invention can also contain further polymerizable monomers (D), preferably likewise (meth)acrylic compounds, that do not correspond to formulae (I) or (II). Preferably, the proportion of the polymerizable monomers (D) is in the range from 0% to 50% by weight, preferably in the range from 0% to 30% by weight, particularly preferably in the range from 0% to 10% by weight, based in each case on the total mass of the polymerizable composition. In this way it is ensured that the synergistic effect of the (meth)acrylic compounds (I) and (II) comes into play. Preferably, the proportion of the polymerizable monomers (D) is less than 50% by weight, preferably less than 30% by weight, particularly preferably less than 10% by weight, based in each case on the total mass of the polymerizable composition.

Preferred monomers (D) are butane-1,3-diol di(meth)acrylate, butane-1,4-diol di(meth)acrylate, hexane-1,6-diol di(meth)acrylate, decane-1,10-diol di(meth)acrylate, dodecane-1,12-diol di(meth)acrylate, cyclohexane-1,4-diol di(meth)acrylate, isobornyl (meth)acrylate, 2-hydroxypropyl 1,3-di(meth)acrylate, 3-hydroxypropyl 1,2-di(meth)acrylate, neopentyl glycol di(meth)acrylate, trimethylolpropane tri(meth)acrylate, pentaerythritol di(meth)acrylate, pentaerythritol tri(meth)acrylate, pentaerythritol tetra(meth)acrylate, 2,2-bis[4-[3-(meth)acryloyloxy-2-hydroxypropoxy]phenyl]propane, 3,14-dioxa-4,13-dioxo-5,12-diazahexadecane 1,16-diyldi(meth)acrylate, 7,7,9-trimethyl-3,14-dioxa-4,13-dioxo-5,12-diazahexadecane 1,16-diyldi(meth)acrylate, 7,9,9-trimethyl-3,14-dioxa-4,13-dioxo-5,12-diazahexadecane 1,16-diyldi(meth)acrylate, 1,5,5-trimethyl-1-[(2-(meth)acryloyloxyethyl)carbamoylmethyl]-3-(2-(meth)acryloyloxyethyl)carbamoylcyclohexane (CAS 42405-01-6 methacrylate; CAS 42404-50-2 acrylate), 1,1′-[methylenebis(4,1-phenyleneiminocarbonyloxy-2,1-ethanediyl)] bis(meth)acrylate (CAS 51243-61-9 methacrylate; CAS 69790-08-5 acrylate), 1,1′-[methylenebis(2,1-phenyleneiminocarbonyloxy-2,1-ethanediyl)]bis(meth)acrylate and 3(4),8(9)-bis((meth)acryloyloxymethyl)tricyclo[5.2.1.0^{2,6}]decane.

In a further configuration, the polymerizable composition may also contain, as polymerizable monomers, exclusively (meth)acrylic compounds that correspond to formulae (I) or (II). Such compositions are thus free of further polymerizable monomers (D).

In one aspect, a composition according to the disclosure comprises 0.01% to 5% by weight, preferably 0.10% to 5% by weight, particularly preferably 0.5% to 4% by weight, based in each case on the total mass of the polymerizable composition, of an initiator or initiator system for the free-radical polymerization. Preferably, the initiator (C) is a photoinitiator. Photoinitiators that may be used are those compounds suitable and customary for (meth)acrylic systems. Advantageously used are alpha-diketones, benzoin alkyl ethers, thioxanthones, benzophenones, acetophenones, acylphosphine oxides or acylgermanium compounds. Preference is given to using monoacylphosphine oxides or bisacylphosphine oxides.

In a preferred configuration, (C) is selected from the group consisting of alpha-diketones, benzoin alkyl ethers, thioxanthones, benzophenones, acetophenones, acylphosphine oxides and acylgermanium compounds, preferably selected from the group consisting of 1-hydroxycyclohexylbenzophenone, 4-(2-hydroxyethoxy)phenyl 2-hydroxy-2-propyl ketone, 2-hydroxy-2-methylpropiophenone, 2,4,6-trimethylbenzoyldiphenylphosphine oxide, phenylbis(2,4,6-trimethylbenzoyl)phosphine oxide, ethyl phenyl(2,4,6-trimethylbenzoyl)phosphinate (CAS 84434-11-7) and (poly(oxy-1,2-ethanediyl),α,α′,α″-1,2,3-propanetriyltris[ω-[[phenyl(2,4,6-trimethylbenzoyl)phosphinyl]oxy] polymer) (CAS 1834525-17-5), particularly preferably selected from the group consisting of 2,4,6-trimethylbenzoyldiphenylphosphine oxide, phenylbis(2,4,6-trimethylbenzoyl)phosphine oxide, ethyl phenyl(2,4,6-trimethylbenzoyl)phosphinate and (poly(oxy-1,2-ethanediyl),α,α′,α″-1,2,3-propanetriyltris[ω-[[phenyl(2,4,6-trimethylbenzoyl)phosphinyl]oxy] polymer).

In a preferred embodiment, a composition according to the disclosure comprises 0.001% to 1% by weight, preferably 0.01% to 0.5% by weight, particularly preferably 0.01% to 0.3% by weight, based in each case on the total mass of the polymerizable composition, of one or more stabilizers (E).

Preferably, (E) is selected from the group consisting of hydroquinone, hydroquinone monomethyl ether, 2,6-di-tert-butyl-4-methylphenol, tert-butylhydroxyanisole and 2,2,6,6-tetramethylpiperidine 1-oxyl.

In order to adjust the color of the material for the desired application, a composition according to the invention may additionally contain one or more colorants (F). Colorants that can be used are inorganic color pigments and organic color pigments or dyes. For tooth-colored applications, yellow, red and brown colorants are used. For dentures, red colorants are used. For models, beige, brown or gray colorants are used. For trays, green or blue colorants may also be used.

An advantage of the compositions according to the disclosure is that the opacity arises already as a result of the polymerization, and thus the use of white pigments can essentially be dispensed with. Only a very small amount of colorants is therefore needed.

In a preferred embodiment, a composition according to the disclosure comprises 0.0001% to 1% by weight, preferably 0.0001% to 0.5% by weight, particularly preferably 0.0001% to 0.10% by weight, based in each case on the total mass of the polymerizable composition, of one or more colorants (F).

In a preferred embodiment, a composition according to the disclosure comprises:
A) in an amount of from 10% to 70% by weight, preferably 10% to 60% by weight,
B) in an amount of from 20% to 85% by weight, preferably 30% to 85% by weight, and
C) in an amount of from 0.1% to 5% by weight, preferably 0.5% to 4% by weight, based in each case on the total mass of the polymerizable composition.

In a further preferred embodiment, a composition according to the disclosure comprises:
A) in an amount of from 10% to 70% by weight, preferably 10% to 60% by weight,
B) in an amount of from 20% to 85% by weight, preferably 30% to 85% by weight,
C) in an amount of from 0.1% to 5% by weight, preferably 0.5% to 4% by weight,
D) in an amount of from 0% to 30% by weight, preferably 0% to 10% by weight, and
E) in an amount of from 0.01% to 0.5% by weight, preferably 0.01% to 0.3% by weight, based in each case on the total mass of the polymerizable composition.

In a further preferred embodiment, a composition according to the disclosure comprises:
A) in an amount of from 10% to 70% by weight, preferably 10% to 60% by weight,
B) in an amount of from 20% to 85% by weight, preferably 30% to 85% by weight,
C) in an amount of from 0.1% to 5% by weight, preferably 0.5% to 4% by weight,
D) in an amount of from 0% to 30% by weight, preferably 0% to 10% by weight,
E) in an amount of from 0.01% to 0.5% by weight, preferably 0.01% to 0.3% by weight, and
F) in an amount of from 0.0001% to 0.5% by weight, preferably 0.0001% to 0.10% by weight,

based in each case on the total mass of the polymerizable composition.

As described above, the interplay between the monomers (A) and (B) results in a printing resin that on polymerization exhibits a change from an essentially transparent monomer mixture to an essentially opaque polymer, such that the advantages described come into effect.

In a preferred embodiment, a free-radically polymerizable composition according to the disclosure prior to the polymerization has an opacity of less than 10% and after the polymerization has an opacity of greater than 50%.

In a particularly preferred embodiment, a free-radically polymerizable composition according to the disclosure prior to the polymerization has an opacity of less than 5%, preferably of less than 3%, and after the polymerization has an opacity of greater than 60%, preferably of greater than 70%.

Preference is given to a free-radically polymerizable composition which prior to the polymerization under the specified measurement conditions has an opacity of <10%, preferably <5%, particularly preferably <3%, and very particularly preferably <1%. Accordingly, such a composition has a translucency of >90%, preferably ≥95%, particularly preferably ≥97%, and very particularly preferably ≥99%.

Preference is given to a free-radically polymerizable composition which after to the polymerization under the specified measurement conditions has an opacity of >50%, preferably ≥60%, and particularly preferably ≥70%.

Also in accordance with the disclosure is a free-radically polymerizable composition as described above, for use in stereolithography and/or 3D printing, preferably for the 3D printing of dental shaped components, with preference for the 3D printing of dental models, trays, try-ins or dentures.

Also in accordance with the disclosure is the use of a free-radically polymerizable composition as described above in stereolithography and/or 3D printing, preferably for the 3D printing of dental shaped components, with preference for the 3D printing of dental models, trays, try-ins or dentures.

### Embodiments

Overall, the invention can be summarized on the basis of the following aspects.

1. Free-radically polymerizable composition comprising
A) 10% to 80% by weight of polymerizable (meth)acrylic compounds of formula I

Y—O—[(C₃H₆O)ᵥ-L¹]ₙ—(C₃H₆O)_{w}—Z (I)

where
Y=H₂C=C(R¹)—C(═O)— or H₂C=C(R¹)—C(═O)—X—L²_
R¹=H or CH₃,
X=O or NH,
L¹=divalent organic linker group,
L²=divalent organic linker group,
Z=Y, H or C1 to C4 alkyl,
v=36 to 100,
w=36 to 200,
v+w=75 to 200,
n=0 to 3,
B) 15% to 85% by weight of polymerizable (meth)acrylic compounds of formula II

{H₂C=C(R¹)—C(═O)—X—[CH₂CH(R²)O]ₘ}_{q}Q (II)

where
R¹=H or CH₃,
R²=H or CH₃,
X=O or NH,
Q=a q-valent organic radical,
m=1 to 20,
q=1 to 4, and
C) 0.01% to 5% by weight of an initiator or initiator system for the free-radical polymerization,
based in each case on the total mass of the polymerizable composition.

2. Free-radically polymerizable composition according to Aspect 1, comprising (A) in an amount of from 10% to 70% by weight, preferably 10% to 60% by weight, particularly preferably 10% to 50% by weight, based on the total mass of the polymerizable composition.

3. Free-radically polymerizable composition according to any of the preceding aspects, wherein in the (meth)acrylic compound of formula I X=O, Z=Y, n=0 and w=75 to 180, preferably 75 to 150, particularly preferably 100 to 150 and very particularly preferably 103 to 121.

4. Free-radically polymerizable composition according to any of the preceding aspects, wherein in the (meth)acrylic compound of formula I X=O, Z=Y, n=1 to 3, v=36 to 100, preferably 36 to 90, particularly preferably 36 to 80, w=36 to 100, preferably 36 to 90, particularly preferably 36 to 80, and v+w=75 to 180, preferably 75 to 150, particularly preferably 100 to 150.

5. Free-radically polymerizable composition according to any of the preceding aspects, wherein in the (meth)acrylic compound of formula I X=O, Z=Y, n=0 to 3, v=36 to 100, preferably 36 to 90, particularly preferably 36 to 80, w=36 to 100, preferably 36 to 90, particularly preferably 36 to 80, and n*v+w=75 to 200, preferably 75 to 180, particularly preferably 100 to 150.

6. Free-radically polymerizable composition according to any of the preceding aspects, wherein, in the (meth)acrylic compound of formula I,

L¹ is a straight-chain, branched and/or cyclic alkylene, arylene, arylalkylene or alkylarylene group, which can contain —O—, —OC(═O)—, —C(═O)O—, —NHC(═O)—, —C(═O)NH—, —OC(═O)NH—, or —NHC(═O)O—, and

L² is a straight-chain, branched and/or cyclic alkylene, arylene, arylalkylene or alkylarylene group, which can contain —O—, —OC(═O)—, —C(═O)O—, —NHC(═O)—, —C(═O)NH—, —OC(═O)NH—, or —NHC(═O)O—.

7. Free-radically polymerizable composition according to any of the preceding aspects, wherein L¹ and/or L² is/are selected from the group consisting of

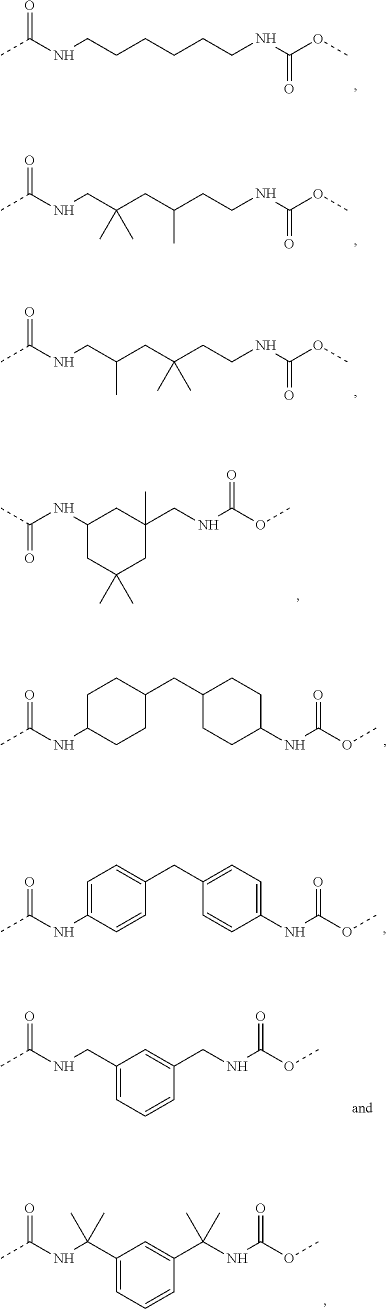

preferably selected from the group consisting of

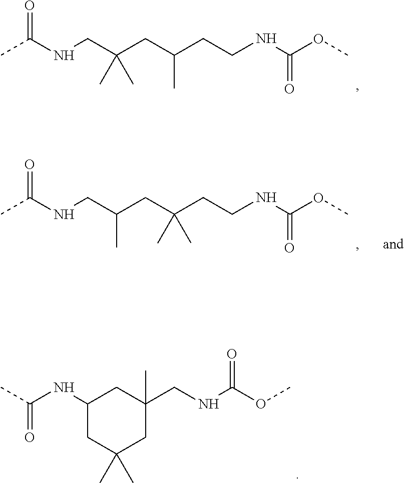

8. Free-radically polymerizable composition according to any of the preceding aspects, wherein L¹ and/or L² is/are selected from the group consisting of

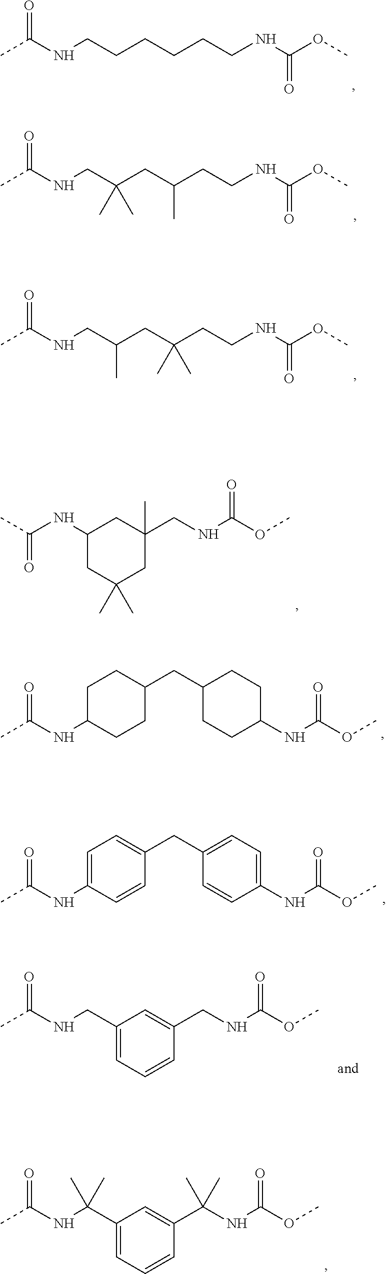

preferably selected from the group consisting of

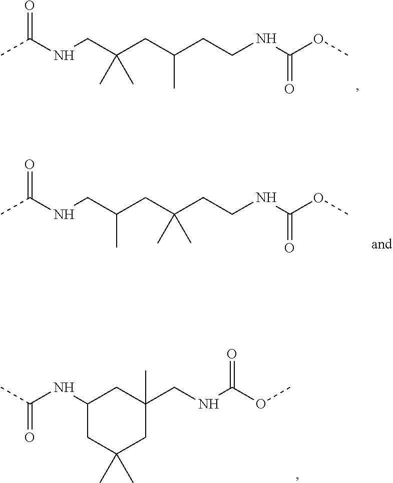

where L¹ is bonded by the left-hand dashed line to the outer oxygen atom of the polypropylene group (C₃H₆O)˜ and is bonded by the right-hand dashed line to the outer carbon atom of the polypropylene group (C₃H₆O)_{W}, and/or L² is bonded by the left-hand dashed line to X and is bonded by the right-hand dashed line, via an oxygen atom (—O—), to the outer carbon atom of one of the polypropylene groups (C₃H₆O), or (C₃H₆O)ᵥ.

9. Free-radically polymerizable composition according to any of the preceding aspects, wherein the (meth)acrylic compound of formula I has a molecular weight of greater than 5000 g/mol, preferably of greater than 6000 g/mol, particularly preferably of greater than 7000 g/mol, and/or in the range from 5000 to 15 000 g/mol, preferably in the range from 6000 to 12 000 g/mol, particularly preferably in the range from 7000 to 10 000 g/mol.

10. Free-radically polymerizable composition according to any of the preceding aspects, wherein the groups (C₃H₆O)ᵥ and (C₃H₆O)_{w} are polypropylene glycol groups, preferably 1,2-polypropylene glycol groups.

11. Free-radically polymerizable composition according to any of the preceding aspects, comprising (B) in an amount of from 20% to 85% by weight, preferably 30% to 85% by weight, particularly preferably 40% to 85% by weight, based on the total mass of the polymerizable composition.

12. Free-radically polymerizable composition according to any of the preceding aspects, wherein in the (meth)acrylic compound of formula II X=O and/or R²=H.

13. Free-radically polymerizable composition according to any of the preceding aspects, wherein, in the (meth)acrylic compound of formula II,

Q=H, C₁ to C₄ alkyl or phenyl, when q=1 or

Q=—(CH₂CH₂)—, —(CH₂CH(CH₃))—,

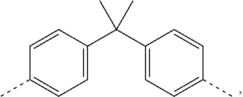

when q=2 or

Q=

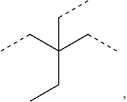

when q=3 or

Q=

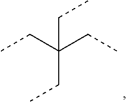

when q=4.

14. Free-radically polymerizable composition according to any of the preceding aspects, wherein the (meth)acrylic compounds of formula (II) with q=1 are selected from the group consisting of 2-hydroxyethyl (meth)acrylate, 2-hydroxypropyl (meth)acrylate, 2-(2-hydroxyethoxy)ethyl (meth)acrylate, 2-[2-(2-hydroxyethoxy)ethoxy]ethyl (meth)acrylate, ethoxylated 2-hydroxyethyl (meth)acrylate, 2-phenoxyethyl (meth)acrylate, 2-(2-phenoxyethoxy)ethyl (meth)acrylate, 2-[2-(2-phenoxyethoxy)ethoxy]ethyl (meth)acrylate and ethoxylated 2-phenoxyethyl (meth)acrylate.

15. Free-radically polymerizable composition according to any of the preceding aspects, wherein the (meth)acrylic compounds of formula (II) with q=2 are selected from the group consisting of ethylene glycol di(meth)acrylate, diethylene glycol di(meth)acrylate, triethylene glycol di(meth)acrylate, tetraethylene glycol di(meth)acrylate, polyethylene glycol di(meth)acrylate, propylene glycol di(meth)acrylate, dipropylene glycol di(meth)acrylate, tripropylene glycol di(meth)acrylate, tetrapropylene glycol di(meth)acrylate, polypropylene glycol di(meth)acrylate, ethoxylated bisphenol A di(meth)acrylate and propoxylated bisphenol A di(meth)acrylate.

16. Free-radically polymerizable composition according to any of the preceding aspects, wherein the (meth)acrylic compounds of formula (II) with q=3 are selected from the group consisting of ethoxylated trimethylolpropane tri(meth)acrylate and propoxylated trimethylolpropane tri(meth)acrylate.

17. Free-radically polymerizable composition according to any of the preceding aspects, wherein the (meth)acrylic compounds of formula (II) with q=4 are selected from the group consisting of ethoxylated pentaerythritol tetra(meth)acrylate and propoxylated pentaerythritol tetra(meth)acrylate.

18. Free-radically polymerizable composition according to any of the preceding aspects, wherein the alkylene oxide units —[CH₂CH(R²)O]ₘ with R²=H or methyl make up at least 30% by weight of the molecular weight of the (meth)acrylic compound of formula II.

19. Free-radically polymerizable composition according to any of the preceding aspects, comprising (meth)acrylic compounds (D) that do not correspond to formulae (I) or (II).

20. Free-radically polymerizable composition according to any of the preceding aspects, comprising (meth)acrylic compounds (D) that do not correspond to formulae (I) or (II) in an amount in the range from 0% to 50% by weight, preferably in the range from 0% to 30% by weight, particularly preferably in the range from 0% to 10% by weight, based in each case on the total mass of the polymerizable composition.

21. Free-radically polymerizable composition according to any of the preceding aspects, comprising (meth)acrylic compounds (D) that do not correspond to formulae (I) or (II) in an amount of less than 50% by weight, preferably less than 30% by weight, particularly preferably of less than 10% by weight, based in each case on the total mass of the polymerizable composition.

22. Free-radically polymerizable composition according to any of the preceding aspects, wherein (D) is selected from the group consisting of butane-1,3-diol di(meth)acrylate, butane-1,4-diol di(meth)acrylate, hexane-1,6-diol di(meth)acrylate, decane-1,10-diol di(meth)acrylate, dodecane-1,12-diol di(meth)acrylate, cyclohexane-1,4-diol di(meth)acrylate, isobornyl (meth)acrylate, 2-hydroxypropyl 1,3-di(meth)acrylate, 3-hydroxypropyl 1,2-di(meth)acrylate, neopentyl glycol di(meth)acrylate, trimethylolpropane tri(meth)acrylate, pentaerythritol di(meth)acrylate, pentaerythritol tri(meth)acrylate, pentaerythritol tetra(meth)acrylate, 2,2-bis[4-[3-(meth)acryloyloxy-2-hydroxypropoxy]phenyl]propane, 3,14-dioxa-4,13-dioxo-5,12-diazahexadecane 1,16-diyldi(meth)acrylate, 7,7,9-trimethyl-3,14-dioxa-4,13-dioxo-5,12-diazahexadecane 1,16-diyldi(meth)acrylate, 7,9,9-trimethyl-3,14-dioxa-4,13-dioxo-5,12-diazahexadecane 1,16-diyldi(meth)acrylate, 1,5,5-trimethyl-1-[(2-(meth)acryloyloxyethyl)carbamoylmethyl]-3-(2-(meth)acryloyloxyethyl)carbamoylcyclohexane (CAS 42405-01-6 methacrylate; CAS 42404-50-2 acrylate), 1,1′-[methylenebis(4,1-phenyleneiminocarbonyloxy-2,1-ethanediyl)] bis(meth)acrylate (CAS 51243-61-9 methacrylate; CAS 69790-08-5 acrylate), 1,1′-[methylenebis(2,1-phenyleneiminocarbonyloxy-2,1-ethanediyl)]bis(meth)acrylate and 3(4),8(9)-bis((meth)acryloyloxymethyl)tricyclo[5.2.1.0^{2,6}]decane.

23. Free-radically polymerizable composition according to any of the preceding aspects, not comprising any (meth)acrylic compounds (D) that do not correspond to formulae (I) or (II).

24. Free-radically polymerizable composition according to any of the preceding aspects, comprising an initiator or initiator system (C) in an amount of from 0.1% to 5% by weight, preferably 0.5% to 4% by weight, based in each case on the total mass of the polymerizable composition.

25. Free-radically polymerizable composition according to any of the preceding aspects, wherein the initiator or the initiator system (C) comprises a photoinitiator or consists of a photoinitiator.

26. Free-radically polymerizable composition according to any of the preceding aspects, wherein (C) is selected from the group consisting of alpha-diketones, benzoin alkyl ethers, thioxanthones, benzophenones, acetophenones, acylphosphine oxides and acylgermanium compounds.

27. Free-radically polymerizable composition according to any of the preceding aspects, wherein (C) is selected from the group consisting of 1-hydroxycyclohexylbenzophenone, 4-(2-hydroxyethoxy)phenyl 2-hydroxy-2-propyl ketone, 2-hydroxy-2-methylpropiophenone, 2,4,6-trimethylbenzoyldiphenylphosphine oxide, phenylbis(2,4,6-trimethylbenzoyl)phosphine oxide, ethyl phenyl(2,4,6-trimethylbenzoyl)phosphinate (CAS 84434-11-7) and (poly(oxy-1,2-ethanediyl),α,α′,α″-1,2,3-propanetriyltris[ω-[[phenyl(2,4,6-trimethylbenzoyl)phosphinyl]oxy] polymer) (CAS 1834525-17-5), particularly preferably selected from the group consisting of 2,4,6-trimethylbenzoyldiphenylphosphine oxide, phenylbis(2,4,6-trimethylbenzoyl)phosphine oxide, ethyl phenyl(2,4,6-trimethylbenzoyl)phosphinate and (poly(oxy-1,2-ethanediyl),α,α′,α″-1,2,3-propanetriyltris[ω-[[phenyl(2,4,6-trimethylbenzoyl)phosphinyl]oxy] polymer).

28. Free-radically polymerizable composition according to any of the preceding aspects, comprising one or more stabilizers (E) in an amount of from 0.001% to 1% by weight, preferably 0.01% to 0.5% by weight, particularly preferably 0.01% to 0.3% by weight, based in each case on the total mass of the polymerizable composition.

29. Free-radically polymerizable composition according to any of the preceding aspects, wherein the stabilizers (E) are selected from the group consisting of hydroquinone, hydroquinone monomethyl ether, 2,6-di-tert-butyl-4-methylphenol, tert-butylhydroxyanisole and 2,2,6,6-tetramethylpiperidine 1-oxyl.

30. Free-radically polymerizable composition according to any of the preceding aspects, comprising one or more colorants (F) in an amount of from 0.0001% to 1% by weight, preferably 0.0001% to 0.5% by weight, particularly preferably 0.0001% to 0.1% by weight, based in each case on the total mass of the polymerizable composition.

31. Free-radically polymerizable composition according to any of the preceding aspects, comprising
A) in an amount of from 10% to 70% by weight, preferably 10% to 60% by weight,
B) in an amount of from 20% to 85% by weight, preferably 30% to 85% by weight, and
C) in an amount of from 0.1% to 5% by weight, preferably 0.5% to 4% by weight, based in each case on the total mass of the polymerizable composition.

32. Free-radically polymerizable composition according to any of the preceding aspects, comprising
A) in an amount of from 10% to 70% by weight, preferably 10% to 60% by weight,
B) in an amount of from 20% to 85% by weight, preferably 30% to 85% by weight,
C) in an amount of from 0.1% to 5% by weight, preferably 0.5% to 4% by weight,
D) in an amount of from 0% to 30% by weight, preferably 0% to 10% by weight, and
E) in an amount of from 0.01% to 0.5% by weight, preferably 0.01% to 0.3% by weight,
based in each case on the total mass of the polymerizable composition.

33. Free-radically polymerizable composition according to any of the preceding aspects, comprising
A) in an amount of from 10% to 70% by weight, preferably 10% to 60% by weight,
B) in an amount of from 20% to 85% by weight, preferably 30% to 85% by weight,
C) in an amount of from 0.1% to 5% by weight, preferably 0.5% to 4% by weight,
D) in an amount of from 0% to 30% by weight, preferably 0% to 10% by weight,
E) in an amount of from 0.01% to 0.5% by weight, preferably 0.01% to 0.3% by weight, and
F) in an amount of from 0.0001% to 0.5% by weight, preferably 0.0001% to 0.1% by weight,
based in each case on the total mass of the polymerizable composition.

34. Free-radically polymerizable composition according to any of the preceding aspects, which prior to the polymerization has an opacity of less than 10% and after the polymerization has an opacity of greater than 50%.

35. Free-radically polymerizable composition according to any of the preceding aspects, which prior to the polymerization has an opacity of less than 5%, preferably of less than 3%, and after the polymerization has an opacity of greater than 60%, preferably of greater than 70%.

36. Free-radically polymerizable composition according to any of the preceding aspects, which prior to the polymerization, preferably under the specified measurement conditions, has an opacity of <10%, preferably <5%, particularly preferably <3%, and very particularly preferably <1%.

37. Free-radically polymerizable composition according to any of the preceding aspects, which prior to the polymerization, preferably under the specified measurement conditions, has a translucency of ≥90%, preferably ≥95%, particularly preferably ≥97%, and very particularly preferably ≥99%.

38. Free-radically polymerizable composition according to any of the preceding aspects, which after the polymerization, preferably under the specified measurement conditions, has an opacity of >50%, preferably >60%, and particularly preferably >70%.

39. Free-radically polymerizable composition according to any of the preceding aspects, for use in stereolithography and/or 3D printing, preferably for the 3D printing of dental shaped components, with preference for the 3D printing of dental models, trays, try-ins or dentures.

40. Use of a free-radically polymerizable composition, according to any of the preceding aspects, in stereolithography and/or 3D printing, preferably for the 3D printing of dental shaped components, with preference for the 3D printing of dental models, trays, try-ins or dentures.

The invention is defined in the appended patent claims. Preferred aspects of the present invention moreover emerge from the following description, including the examples.

Insofar as configurations defined for one aspect according to the invention (composition or use) are indicated as preferred, the corresponding statements also respectively apply for the other aspects of the present invention, mutatis mutandis. Preferred individual features of aspects according to the invention (as defined in the claims and/or disclosed in the description) are combinable with one another and are preferably combined with one another unless in the individual case the present text indicates otherwise to the person skilled in the art.

### Examples

The aims and advantages of the present disclosure are further illustrated by the examples which follow; however, the specific materials and the amounts thereof that are mentioned in these examples, as well as other conditions and details, are not to be interpreted such as to disproportionately limit the present disclosure.

The materials used in the examples are summarized below.
PPG: polypropylene glycol (CAS 25322-69-4)
PEG: polyethylene glycol (CAS 25322-68-3)
HMDI: hexamethylene diisocyanate (CAS 822-06-0)
TMDI: 2,2,4-trimethylhexamethylene diisocyanate/2,4,4-trimethylhexamethylene diisocyanate (isomer mixture; CAS 32052-51-0)
IPDI: isophorone diisocyanate (CAS 4098-71-9)
DBTL: dibutyltin dilaurate (CAS 77-58-7)
BHT: 2,6-di-tert-butyl-4-methylphenol (CAS 128-37-0)
HEMA: 2-hydroxyethyl methacrylate (CAS 868-77-9)
HEA: 2-hydroxyethyl acrylate (CAS 818-61-1)
PheMA: 2-phenoxyethyl methacrylate (CAS 10595-06-9)
PheA-4: ethoxylated 2-phenoxyethyl acrylate (4 EO units; CAS 56641-05-5)
TEDMA: triethylene glycol dimethacrylate (CAS 109-16-0)
PEG600DMA: polyethylene glycol 600 dimethacrylate (CAS 25852-47-5)
BisEMA-6: ethoxylated bisphenol A dimethacrylate (6 EO units; CAS 41637-38-1)
BisEMA-10: ethoxylated bisphenol A dimethacrylate (10 EO units; CAS 41637-38-1)
TMPTA-9: ethoxylated trimethylolpropane triacrylate (9 EO units; CAS 28961-43-5)
PETA-4: ethoxylated pentaerythritol tetraacrylate (4 EO units; CAS 51728-26-8)
IBOMA: isobornyl methacrylate (CAS 7534-94-3)
IBOA: isobornyl acrylate (CAS 5888-33-5)
THFMA: tetrahydrofurfuryl methacrylate (CAS 2455-24-5)
ACMO: acryloylmorpholine (CAS 5117-12-4)
TMPTA: trimethylolpropane triacrylate (CAS 15625-89-5)
PETA: pentaerythritol tetraacrylate (CAS 4986-89-4)
HDDMA: hexane-1,6-diol dimethacrylate (CAS 6606-59-3)
TCDDMA: 3(4),8(9)-bis(methacryloyloxymethyl)tricyclo[5.2.1.0^{2,6}]decane (CAS 43048-08-4)
UDMA: 7,7,9-trimethyl-3,14-dioxa-4,13-dioxo-5,12-diazahexadecane 1,16-diyldi(meth)acrylate/7,9,9-trimethyl-3,14-dioxa-4,13-dioxo-5,12-diazahexadecane 1,16-diyldi(meth)acrylate (isomer mixture; CAS 72869-86-4)
MonoUDA: 2-[[(butylamino)carbonyl]oxy]ethyl acrylate (CAS 63225-53-6)
CTFA: (5-ethyl-1,3-dioxan-5-yl)methyl acrylate (CAS 66492-51-1)
IDMA: isodeycl methacrylate (CAS 29964-84-9)
TPO: Diphenyl(2,4,6-trimethylbenzoyl)phosphine oxide (CAS 75980-60-8)

### Synthesis of the Polypropylene Glycol (Meth)Acrylic Compounds

### Example 1a (PPG 5000 Dimethacrylate)

100.00 g of PPG 5000 and 4.05 g of triethylamine are dissolved in 400 ml of chloroform. 4.18 g of methacryloyl chloride are dissolved in 20 ml of chloroform and slowly metered in at 0° C. After addition has ended, stirring is continued at 0° C. for one hour. Thereafter, the mixture is slowly warmed to room temperature and stirred at room temperature for a further 18 hours. The precipitate formed is filtered off and the filtrate is washed twice with water, three times with 10% hydrochloric acid, and twice with saturated sodium chloride solution. The organic phase is dried over magnesium sulfate. 50 mg of BHT are added and the solvent is removed under reduced pressure. A light yellow liquid is obtained.

### Example 1b (PPG 6000 Dimethacrylate)

120.00 g of PPG 6000 and 4.05 g of triethylamine are dissolved in 400 ml of chloroform. 4.18 g of methacryloyl chloride are dissolved in 25 ml of chloroform and slowly metered in at 0° C. After addition has ended, stirring is continued at 0° C. for one hour. Thereafter, the mixture is slowly warmed to room temperature and stirred at room temperature for a further 18 hours. The precipitate formed is filtered off and the filtrate is washed twice with water, three times with 10% hydrochloric acid, and twice with saturated sodium chloride solution. The organic phase is dried over magnesium sulfate. 50 mg of BHT are added and the solvent is removed under reduced pressure. A light yellow liquid is obtained.

### Example 1c (PPG 5000 Urethane Dimethacrylate)

200.00 g of PPG 5000 are dissolved in 400 ml of chloroform. Then, 6.21 g of 2-isocyanatoethyl methacrylate, 200 mg of dibutyltin dilaurate and 100 mg of BHT are added. The mixture is heated to 55° C. with stirring for 6 hours. As reaction control, the disappearance of the NCO band at approx. 2270 cm⁻¹ in the IR spectrum is observed. The solvent is then removed under reduced pressure. A light yellow liquid is obtained. Viscosity (25° C.): 0.5 Pa*s

### Example 1d (PPG 6000 Urethane Dimethacrylate)

240.00 g of PPG 6000 are dissolved in 400 ml of chloroform. Then, 6.21 g of 2-isocyanatoethyl methacrylate, 200 mg of dibutyltin dilaurate and 100 mg of BHT are added. The mixture is heated to 55° C. with stirring for 6 hours. As reaction control, the disappearance of the NCO band at approx. 2270 cm⁻¹ in the IR spectrum is observed. The solvent is then removed under reduced pressure. A light yellow liquid is obtained. Viscosity (25° C.): 0.5 Pa*s

### Example 1e (PPG 5000-HMDI-HEMA)

100.00 g of PPG 5000, 6.73 g of hexamethylene diisocyanate (HMDI) and 5.21 g of 2-hydroxyethyl methacrylate (HEMA) are dissolved in 200 ml of chloroform. Then, 100 mg of dibutyltin dilaurate and 50 mg of BHT are added. The mixture is heated to 55° C. with stirring for 6 hours. As reaction control, the disappearance of the NCO band at approx. 2270 cm⁻¹ in the IR spectrum is observed. The solvent is then removed under reduced pressure. A light yellow liquid is obtained.

### Example 1f (PPG 5000-TMDI-HEA)

100.00 g of PPG 5000, 8.41 g of trimethylhexamethylene diisocyanate (TMDI) and 4.64 g of 2-hydroxyethyl acrylate (HEA) are dissolved in 200 ml of chloroform. Then, 100 mg of dibutyltin dilaurate and 50 mg of BHT are added. The mixture is heated to 55° C. with stirring for 6 hours. As reaction control, the disappearance of the NCO band at approx. 2270 cm⁻¹ in the IR spectrum is observed. The solvent is then removed under reduced pressure. A light yellow liquid is obtained.

### Example 1g (PPG 5000-IPDI-HEMA)

100.00 g of PPG 5000, 8.89 g of isophorone diisocyanate (IPDI) and 5.21 g of 2-hydroxyethyl methacrylate (HEMA) are dissolved in 200 ml of chloroform. Then, 100 mg of dibutyltin dilaurate and 50 mg of BHT are added. The mixture is heated to 55° C. with stirring for 6 hours. As reaction control, the disappearance of the NCO band at approx. 2270 cm⁻¹ in the IR spectrum is observed. The solvent is then removed under reduced pressure. A light yellow liquid is obtained.

### Example 1h (PPG 5000-IPDI-HEA)

100.00 g of PPG 5000, 8.89 g of isophorone diisocyanate (IPDI) and 4.64 g of 2-hydroxyethyl acrylate (HEA) are dissolved in 200 ml of chloroform. Then, 100 mg of dibutyltin dilaurate and 50 mg of BHT are added. The mixture is heated to 55° C. with stirring for 6 hours. As reaction control, the disappearance of the NCO band at approx. 2270 cm⁻¹ in the IR spectrum is observed. The solvent is then removed under reduced pressure. A light yellow liquid is obtained.

### Example 1i (oligo-PPG 5000-IPDI-HEMA)

100.00 g of PPG 3000, 11.12 g of isophorone diisocyanate (IPDI) and 4.38 g of 2-hydroxyethyl methacrylate (HEMA) are dissolved in 200 ml of chloroform. Then, 100 mg of dibutyltin dilaurate and 50 mg of BHT are added. The mixture is heated to 55° C. with stirring for 6 hours. As reaction control, the disappearance of the NCO band at approx. 2270 cm⁻¹ in the IR spectrum is observed. The solvent is then removed under reduced pressure. A light yellow liquid is obtained.

### Comparative Example V1a (PPG 2000 Dimethacrylate)

40.00 g of PPG 2000 and 4.05 g of triethylamine are dissolved in 200 ml of chloroform. 4.18 g of methacryloyl chloride are dissolved in 25 ml of chloroform and slowly metered in at 0° C. After addition has ended, stirring is continued at 0° C. for one hour. Thereafter, the mixture is slowly warmed to room temperature and stirred at room temperature for a further 18 hours. The precipitate formed is filtered off and the filtrate is washed twice with water, three times with 10% hydrochloric acid, and twice with saturated sodium chloride solution. The organic phase is dried over magnesium sulfate. 50 mg of BHT are added and the solvent is removed under reduced pressure. A light yellow liquid is obtained.

### Comparative Example V1b (PPG 1000 Urethane Dimethacrylate)

80.00 g of PPG 1000 are dissolved in 200 ml of chloroform. Then, 12.42 g of 2-isocyanatoethyl methacrylate, 200 mg of dibutyltin dilaurate and 100 mg of BHT are added. The mixture is heated to 55° C. with stirring for 6 hours. As reaction control, the disappearance of the NCO band at approx. 2270 cm⁻¹ in the IR spectrum is observed. The solvent is then removed under reduced pressure. A light yellow liquid is obtained. Viscosity (25° C.): 1.3 Pa*s

### Comparative Example V1c (PPG 4000 Urethane Dimethacrylate)

160.00 g of PPG 4000 are dissolved in 400 ml of chloroform. Then, 12.42 g of 2-isocyanatoethyl methacrylate, 200 mg of dibutyltin dilaurate and 100 mg of BHT are added. The mixture is heated to 55° C. with stirring for 6 hours. As reaction control, the disappearance of the NCO band at approx. 2270 cm⁻¹ in the IR spectrum is observed. The solvent is then removed under reduced pressure. A light yellow liquid is obtained. Viscosity (25° C.): 0.5 Pa*s

### Comparative Example V1d (PPG 1000-TMDI-HEMA)

40.00 g of PPG 1000, 16.82 g of trimethylhexamethylene diisocyanate (TMDI) and 9.29 g of 2-hydroxyethyl acrylate (HEA) are dissolved in 200 ml of chloroform. Then, 100 mg of dibutyltin dilaurate and 50 mg of BHT are added. The mixture is heated to 55° C. with stirring for 6 hours. As reaction control, the disappearance of the NCO band at approx. 2270 cm⁻¹ in the IR spectrum is observed. The solvent is then removed under reduced pressure. A light yellow liquid is obtained.

### Example V1e (PEG 6000 Dimethacrylate)

120.00 g of PEG 6000 and 4.05 g of triethylamine are dissolved in 400 ml of chloroform. 4.18 g of methacryloyl chloride are dissolved in 25 ml of chloroform and slowly metered in at 0° C. After addition has ended, stirring is continued at 0° C. for one hour. Thereafter, the mixture is slowly warmed to room temperature and stirred at room temperature for a further 18 hours. The precipitate formed is filtered off and the filtrate is washed twice with water, three times with 10% hydrochloric acid, and twice with saturated sodium chloride solution. The organic phase is dried over magnesium sulfate. 50 mg of BHT are added and the solvent is removed under reduced pressure. A light yellow liquid is obtained.

### Measurement Methods:

Flexural strength (FS): The flexural strengths were determined in a manner adapted to ISO 4049:2009. Test specimens having the dimensions 40 mm×5 mm×5 mm were produced from the printing resins by means of 3D printing (SolFlex350, W2P Engineering GmbH; wavelength 385 nm, power 8.3 mW/cm², pixel size 50 μm, layer thickness 50 μm) and were post-exposed with an Otoflash (VOCO GmbH) using 2×2000 flashes. The flexural strength is determined at a travel rate of 1 mm/min on a Zwick universal tester (Zwick GmbH & Co. KG, Ulm).

Opacity (solid, polymerized samples): To determine the opacity, test specimens having a radius of 10 mm and a thickness of 2.3 mm were printed from the printing resins (SolFlex350, W2P Engineering GmbH; wavelength 385 nm, power 8.3 mW/cm², pixel size 50 μm, layer thickness 50 μm). The test specimens were sanded to 2.0 mm using 2500 and 4000 grit sandpaper. To determine the opacity, the test specimens produced were analysed using a Hunterlab ColorFlex EZ 45/0 dual-beam spectrophotometer. The analysis was effected under standard illuminant D65 and at a 10° observer angle in reflection mode against a black tile and a white tile.

Opacity (liquid samples): The determination of the opacity of the liquid resins was likewise conducted using a Hunterlab ColorFlex EZ 45/0 dual-beam spectrophotometer. In order to preclude a premature polymerization as a result of the light used, the liquid resins were measured without photoinitiators. The photoinitiators were only added subsequently. For the measurement, the resins were filled into a glass cuvette (base thickness 2.0 mm; filling level 8.0 mm). The analysis was likewise effected under standard illuminant D65 and at a 10° observer angle in reflection mode against a black tile and a white tile.

The L*a*b* values originate from the measurement against the white tile. To determine the opacity and translucency, the tristimulus values Y_{black} und Y_{white} from the measurement against the black tile and the white tile, respectively, were used. The opacity in percent is calculated according to the formula

Opacity [%]=100×Y_{black}/Y_{white}.

Viscosity: Measured as standard on an Anton Paar Physica MCR 301 rheometer with a 50 mm measurement plate (plate/plate), 0.5 mm plate distance and 1 g of substance. Prior to the measurement, the plate is adjusted to a temperature of 25° C. The measurement duration is 30 s with a shear rate of 10/s.

Molecular weight: The molecular weight was determined with the aid of GPC (Agilent Technologies 1260 Infinity). The system is composed of a precolumn (length 50 mm, diameter 8 mm, particle size 5 μm) and two main columns (I: length 300 mm, diameter 8 mm, particle size 5 μm, 50 A; II: length 300 mm, diameter 8 mm, particle size 5 μm, 100 A). The eluent used is THF. The standard used is polystyrene.

### Working Examples

The monomers listed in Tables 1 to 17 were each weighed out in the amounts indicated and stirred at room temperature with a precision glass stirrer for 60 minutes, until a homogeneous solution had formed. The opacity of these liquid solutions was then measured. Thereafter, the photoinitiators were added and stirring was continued with a precision glass stirrer at room temperature for a further 60 minutes until the photoinitiators had completely dissolved.

TABLE 1

| | | Example | | | | | |
|---|---|---|---|---|---|---|---|
| | | 2a | 2b | 2c | 2d | 2e | 2f |
| (A) | Monomer 1a | 14.25 | 29.25 | 49.25 | 14.25 | 29.25 | 49.25 |
| (B) | HEMA | 84.25 | 69.25 | 49.25 | — | — | — |
| | HEA | — | — | — | 84.25 | 69.25 | 49.25 |
| (C) | TPO | 1.50 | 1.50 | 1.50 | 1.50 | 1.50 | 1.50 |

| | | | | | | |
|---|---|---|---|---|---|---|
| Opacity (monomer) | 0 | 0 | 0 | 0 | 0 | 0 |
| Opacity (polymer) | 76 | 80 | 77 | 74 | 74 | 64 |

TABLE 2

| Example | | 2g | 2h | 2i | 2j | 2k | 2l |
|---|---|---|---|---|---|---|---|
| (A) | Monomer 1a | 14.25 | 29.25 | 49.25 | 14.25 | 29.25 | 49.25 |
| (B) | PheMA | 84.25 | 69.25 | 49.25 | — | — | — |
| | PheA-4 | — | — | — | 84.25 | 69.25 | 49.25 |
| (C) | TPO | 1.50 | 1.50 | 1.50 | 1.50 | 1.50 | 1.50 |

| | | | | | | |
|---|---|---|---|---|---|---|
| Opacity (monomer) | 0 | 0 | 0 | 0 | 0 | 0 |
| Opacity (polymer) | 81 | 90 | 84 | 73 | 71 | 62 |

TABLE 3

| Example | | 2m | 2n | 2o | 2p | 2q | 2r |
|---|---|---|---|---|---|---|---|
| (A) | Monomer 1a | 14.25 | 29.25 | 49.25 | 14.25 | 29.25 | 49.25 |
| (B) | TEDMA | 84.25 | 69.25 | 49.25 | — | — | — |
| | PEG600DMA | — | — | — | 84.25 | 69.25 | 49.25 |
| (C) | TPO | 1.50 | 1.50 | 1.50 | 1.50 | 1.50 | 1.50 |

| | | | | | | |
|---|---|---|---|---|---|---|
| Opacity (monomer) | 0 | 0 | 0 | 0 | 0 | 0 |
| Opacity (polymer) | 72 | 85 | 84 | 71 | 82 | 81 |

TABLE 4

| Example | | 2s | 2t | 2u | 2v | 2w | 2x |
|---|---|---|---|---|---|---|---|
| (A) | Monomer 1a | 14.25 | 29.25 | 49.25 | 14.25 | 29.25 | 49.25 |
| (B) | BisEMA-6 | 84.25 | 69.25 | 49.25 | — | — | — |
| | BisEMA-10 | — | — | — | 84.25 | 69.25 | 49.25 |
| (C) | TPO | 1.50 | 1.50 | 1.50 | 1.50 | 1.50 | 1.50 |

| | | | | | | |
|---|---|---|---|---|---|---|
| Opacity (monomer) | 0 | 0 | 0 | 0 | 0 | 0 |
| Opacity (polymer) | 78 | 89 | 75 | 80 | 92 | 92 |

TABLE 5

| Example | | 3a | 3b | 3c | 3d | 3e | 3f |
|---|---|---|---|---|---|---|---|
| (A) | Monomer 1a | 14.25 | 29.25 | 49.25 | 14.25 | 29.25 | 49.25 |
| (B) | TMPTA-9 | 84.25 | 69.25 | 49.25 | — | — | — |
| | PETA-4 | — | — | — | 84.25 | 69.25 | 49.25 |
| (C) | TPO | 1.50 | 1.50 | 1.50 | 1.50 | 1.50 | 1.50 |

| | | | | | | |
|---|---|---|---|---|---|---|
| Opacity (monomer) | 0 | 0 | 0 | 0 | 0 | 0 |
| Opacity (polymer) | 72 | 77 | 81 | 63 | 76 | 81 |

TABLE 6

| Example | | 3g | 3h | 3i | 3j | 3k | 3l |
|---|---|---|---|---|---|---|---|
| (A) | Monomer 1a | 14.50 | 24.50 | 34.50 | 14.50 | 24.50 | 34.50 |
| (B) | HEMA | 37.25 | 32.25 | 27.25 | 32.25 | 27.25 | 22.25 |
| | TEDMA | 37.25 | 32.25 | 27.25 | 32.25 | 27.25 | 22.25 |
| (D) | IBOMA | 9.50 | 9.50 | 9.50 | 19.50 | 19.50 | 19.50 |
| (C) | TPO | 1.50 | 1.50 | 1.50 | 1.50 | 1.50 | 1.50 |

| | | | | | | |
|---|---|---|---|---|---|---|
| Opacity (monomer) | 0 | 0 | 0 | 0 | 0 | 0 |
| Opacity (polymer) | 63 | 67 | 66 | 54 | 59 | 58 |

TABLE 7

| Example | | 3m | 3n | 3o | 3p | 3q | 3r |
|---|---|---|---|---|---|---|---|
| (A) | Monomer 1b | 29.25 | — | — | — | — | — |
| | Monomer 1c | — | 29.25 | — | — | — | — |
| | Monomer 1d | — | — | 29.25 | — | — | — |
| | Monomer 1e | — | — | — | 29.25 | — | — |
| | Monomer 1f | — | — | — | — | 29.25 | — |
| | Monomer 1g | — | — | — | — | — | 29.25 |
| (B) | HEMA | 69.25 | 69.25 | 69.25 | 69.25 | 69.25 | 69.25 |
| (C) | TPO | 1.50 | 1.50 | 1.50 | 1.50 | 1.50 | 1.50 |

| | | | | | | |
|---|---|---|---|---|---|---|
| Opacity (monomer) | 0 | 0 | 0 | 0 | | 0 |
| Opacity (polymer) | 83 | 81 | 83 | 82 | 79 | 84 |

TABLE 8

| Example | | 3s | 3t |
|---|---|---|---|
| (A) | Monomer 1h | 29.25 | — |
| | Monomer 1i | — | 29.25 |
| (B) | HEMA | 69.25 | 69.25 |
| (C) | TPO | 1.50 | 1.50 |

| | | |
|---|---|---|
| Opacity (monomer) | 0 | 0 |
| Opacity (polymer) | 83 | 85 |

TABLE 9

| Comparative example | | 4a | 4b | 4c | 4d | 4e | 4f |
|---|---|---|---|---|---|---|---|
| (A) | Monomer 1a | 4.25 | 84.25 | 4.25 | 84.25 | 4.25 | 84.25 |
| (B) | HEMA | 94.25 | 14.25 | — | — | — | — |
| | HEA | — | — | 94.25 | 14.25 | — | — |
| | TEDMA | — | — | — | — | 94.25 | 14.25 |
| (C) | TPO | 1.50 | 1.50 | 1.50 | 1.50 | 1.50 | 1.50 |

| | | | | | | |
|---|---|---|---|---|---|---|
| Opacity (monomer) | 0 | 0 | 0 | 0 | 0 | 0 |
| Opacity (polymer) | 23 | 37 | 18 | 31 | 25 | 38 |

TABLE 10

| Comparative example | | 4g | 4h | 4i | 4j | 4k | 4l |
|---|---|---|---|---|---|---|---|
| (A) | Monomer 1a | 14.25 | 29.25 | 49.25 | 14.25 | 29.25 | 49.25 |
| (D) | IBOMA | 84.25 | 69.25 | 49.25 | — | — | — |
| | IBOA | — | — | — | 84.25 | 69.25 | 49.25 |
| (C) | TPO | 1.50 | 1.50 | 1.50 | 1.50 | 1.50 | 1.50 |

| | | | | | | |
|---|---|---|---|---|---|---|
| Opacity (monomer) | 0 | 0 | 0 | 0 | 0 | 0 |
| Opacity (polymer) | 6 | 12 | 4 | 1 | 1 | 1 |

TABLE 11

| Comparative example | | 4m | 4n | 4o | 4p | 4q | 4r |
|---|---|---|---|---|---|---|---|
| (A) | Monomer 1a | 14.25 | 29.25 | 49.25 | 14.25 | 29.25 | 49.25 |
| (D) | THFMA | 84.25 | 69.25 | 49.25 | — | — | — |
| | ACMO | — | — | — | 84.25 | 69.25 | 49.25 |
| (C) | TPO | 1.50 | 1.50 | 1.50 | 1.50 | 1.50 | 1.50 |

| | | | | | | |
|---|---|---|---|---|---|---|
| Opacity (monomer) | 0 | 0 | 0 | 0 | 0 | 0 |
| Opacity (polymer) | * | 13 | 1 | 2 | 7 | 12 |

* no curing

TABLE 12

| Comparative example | | 4s | 4t | 4u | 4v | 4w | 4x |
|---|---|---|---|---|---|---|---|
| (A) | Monomer 1a | 14.25 | 29.25 | 49.25 | 14.25 | 29.25 | 49.25 |
| (D) | TMPTA | 84.25 | 69.25 | 49.25 | — | — | — |
| | PETA | — | — | — | 84.25 | 69.25 | 49.25 |
| (C) | TPO | 1.50 | 1.50 | 1.50 | 1.50 | 1.50 | 1.50 |

| | | | | | | |
|---|---|---|---|---|---|---|
| Opacity (monomer) | 0 | 0 | 0 | 0 | 0 | 0 |
| Opacity (polymer) | 2 | 7 | 12 | 13 | 17 | 22 |

TABLE 13

| Comparative example | | 5a | 5b | 5c | 5d | 5e | 5f |
|---|---|---|---|---|---|---|---|
| (A) | Monomer 1a | 14.25 | 29.25 | 49.25 | 14.25 | 29.25 | 49.25 |
| (D) | HDDMA | 84.25 | 69.25 | 49.25 | — | — | — |
| | TCDDMA | — | — | — | 84.25 | 69.25 | 49.25 |
| (C) | TPO | 1.50 | 1.50 | 1.50 | 1.50 | 1.50 | 1.50 |

| | | | | | | |
|---|---|---|---|---|---|---|
| Opacity (monomer) | 0 | 0 | 0 | 0 | 0 | 0 |
| Opacity (polymer) | 4 | 30 | 25 | 1 | 3 | 7 |

TABLE 14

| Comparative example | | 5g | 5h | 5i | 5j | 5k | 5l |
|---|---|---|---|---|---|---|---|
| (A) | Monomer 1a | 14.25 | 29.25 | 49.25 | 14.25 | 29.25 | 49.25 |
| (D) | UDMA | 84.25 | 69.25 | 49.25 | — | — | — |
| | MonoUDA | — | — | — | 84.25 | 69.25 | 49.25 |
| (C) | TPO | 1.50 | 1.50 | 1.50 | 1.50 | 1.50 | 1.50 |

| | | | | | | |
|---|---|---|---|---|---|---|
| Opacity (monomer) | 0 | 0 | 0 | 0 | 0 | 0 |
| Opacity (polymer) | 1 | 1 | 24 | 2 | 2 | 3 |

TABLE 15

| Comparative example | | 5m | 5n | 5o | 5p | 5q | 5r |
|---|---|---|---|---|---|---|---|
| (A) | Monomer 1a | 14.25 | 29.25 | 49.25 | 14.25 | 29.25 | 49.25 |
| (D) | CTFA | 84.25 | 69.25 | 49.25 | — | — | — |
| | IDMA | — | — | — | 84.25 | 69.25 | 49.25 |
| (C) | TPO | 1.50 | 1.50 | 1.50 | 1.50 | 1.50 | 1.50 |

| | | | | | | |
|---|---|---|---|---|---|---|
| Opacity (monomer) | 0 | 0 | 0 | 0 | 0 | 0 |
| Opacity (polymer) | 2 | 2 | 2 | * | * | 5 |

* no curing

TABLE 16

| Comparative example | | 5s | 5t | 5u | 5v | 5w | 5x |
|---|---|---|---|---|---|---|---|
| (A) | Monomer 1a | 9.50 | 9.50 | 9.50 | 9.50 | 9.50 | 9.50 |
| (B) | HEMA | 79.50 | 69.50 | 39.75 | 34.75 | — | — |
| | TEDMA | — | — | 39.75 | 34.75 | 79.50 | 69.50 |
| (D) | IBOMA | 9.50 | 19.50 | 9.50 | 19.50 | 9.50 | 19.50 |
| (C) | TPO | 1.50 | 1.50 | 1.50 | 1.50 | 1.50 | 1.50 |

| | | | | | | |
|---|---|---|---|---|---|---|
| Opacity (monomer) | 0 | 0 | 0 | 0 | 0 | 0 |
| Opacity (polymer) | 47 | 36 | 48 | 36 | 45 | 34 |

TABLE 17

| Comparative example | | 6a | 6b | 6c | 6d | 6e |
|---|---|---|---|---|---|---|
| | Monomer V1a | 29.25 | — | — | — | — |
| | Monomer V1b | — | 29.25 | — | — | — |
| | Monomer V1c | — | — | 29.25 | — | — |
| | Monomer V1d | — | — | — | 29.25 | — |
| | Monomer V1e | — | — | — | — | 29.25 |
| (B) | HEMA | 69.25 | 69.25 | 69.25 | 69.25 | 69.25 |
| (C) | TPO | 1.50 | 1.50 | 1.50 | 1.50 | 1.50 |

| | | | | | |
|---|---|---|---|---|---|
| Opacity (monomer) | 0 | 0 | 0 | 0 | 0 |
| Opacity (polymer) | 27 | 18 | 43 | 19 | 34 |

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A free-radically polymerizable composition comprising
A) 10% to 80% by weight of polymerizable (meth)acrylic compounds of formula I
Y—O—[(C₃H₆O)ᵥ—L¹]ₙ—(C₃H₆O)_{w}—Z (I)
wherein
Y=H₂C=C(R¹)—C(═O)— or H₂C=C(R¹)—C(═O)—X—L²-,
R¹=H or CH₃,
X=O or NH,
L¹=divalent organic linker group,
L²=divalent organic linker group,
Z=Y, H or C1 to C₄ alkyl,
v=36 to 100,
w=36 to 200,
v+w=75 to 200,
n=0 to 3,
B) 15% to 85% by weight of polymerizable (meth)acrylic compounds of formula II
{H₂C=C(R¹)—C(═O)—X—[CH₂CH(R²)O]ₘ}_{q}Q (II)
wherein
R¹=H or CH₃,
R²=H or CH₃,
X=O or NH,
Q=a q-valent organic radical,
m=1 to 20,
q=1 to 4, and
C) 0.01% to 5% by weight of an initiator or initiator system for the free-radical polymerization,
based in each case on the total mass of the polymerizable composition.

2. The free-radically polymerizable composition of claim 1, comprising
A) in an amount of from 10% to 70% by weight,
B) in an amount of from 20% to 85% by weight, and
C) in an amount of from 0.1% to 5% by weight,
based in each case on the total mass of the polymerizable composition.

3. The free-radically polymerizable composition of claim 1, wherein, in the (meth)acrylic compound of formula I,
x=O,
Z=Y,
n=0 and
w=75 to 180.

4. The free-radically polymerizable composition of claim 1, wherein, in the (meth)acrylic compound of formula I,
X=O,
Z=Y,
n=1 to 3,
v=36 to 100,
w=36 to 100, and
v+w=75 to 180.

5. The free-radically polymerizable composition of claim 1, wherein, in the (meth)acrylic compound of formula I,
L¹ is a straight-chain, branched and/or cyclic alkylene, arylene, arylalkylene or alkylarylene group, comprising —O—, —OC(═O)—, —C(═O)O—, —NHC(═O)—, —C(═O)NH—, —OC(═O)NH—, or —NHC(═O)O—, and
L² is a straight-chain, branched and/or cyclic alkylene, arylene, arylalkylene or alkylarylene group, comprising —O—, —OC(═O)—, —C(═O)O—, —NHC(═O)—, —C(═O)NH—, —OC(═O)NH—, or —NHC(═O)O—,

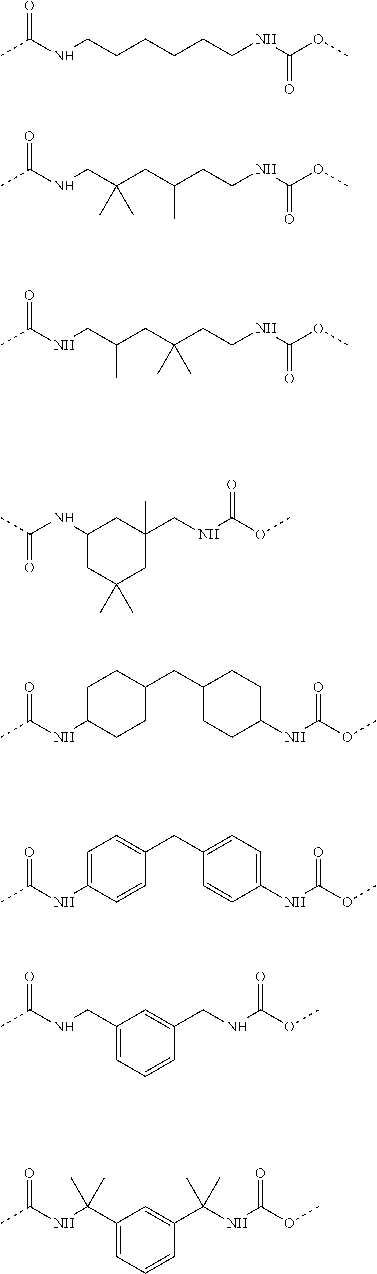

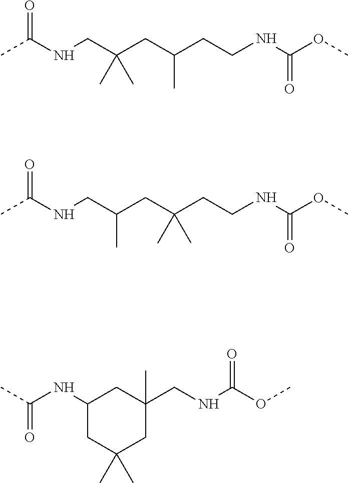

6. The free-radically polymerizable composition of claim 1, wherein the (meth)acrylic compound of formula I has a molecular weight of greater than 5000 g/mol.

7. The free-radically polymerizable composition of claim 1, wherein, in the (meth)acrylic compound of formula II,
X=O and/or
R²=H.

8. The free-radically polymerizable composition of claim 1, wherein, in the (meth)acrylic compound of formula II,
Q=H, C₁ to C₄ alkyl or phenyl, when q=1, or
Q=—(CH₂CH₂)—, —(CH₂CH(CH₃))—, 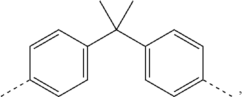
when q=2, or
Q= 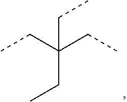
when q=3, or
Q= 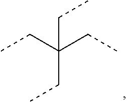
when q=4.

9. The free-radically polymerizable composition of claim 1, additionally comprising
D) polymerizable (meth)acrylic compounds that do not correspond to formulae I or II.

10. The free-radically polymerizable composition of claim 1, which prior to the polymerization has an opacity of less than 10% and after the polymerization has an opacity of greater than 50%.

11. The free-radically polymerizable composition of claim 1, which prior to the polymerization has an opacity of less than 5%, and after the polymerization has an opacity of greater than 60%.

12. Use of a free-radically polymerizable composition, of claim 1, in stereolithography and/or 3D printing, preferably for the 3D printing of dental shaped components, with preference for the 3D printing of dental models, trays, try-ins or dentures.

13. The free-radically polymerizable composition of claim 1, wherein, in the (meth)acrylic compound of formula I, L¹ and/or L² is/are selected from the group consisting of 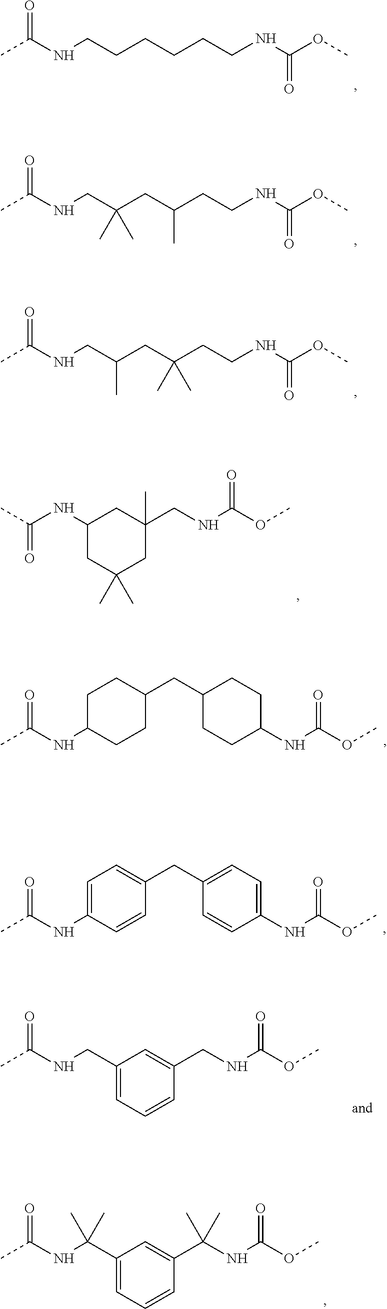
where L¹ is bonded by the left-hand dashed line to the outer oxygen atom of the polypropylene group (C₃H₆O)ᵥ and is bonded by the right-hand dashed line to the outer carbon atom of the polypropylene group (C₃H₆O)_{w}, and/or where L² is bonded by the left-hand dashed line to X and is bonded by the right-hand dashed line, via an oxygen atom (—O—), to the outer carbon atom of one of the polypropylene groups (C₃H₆O)_{w} or (C₃H₆O)ᵥ.

14. The free-radically polymerizable composition of claim 13, wherein, in the (meth)acrylic compound of formula I, L¹ and/or L² is/are selected from the group consisting of

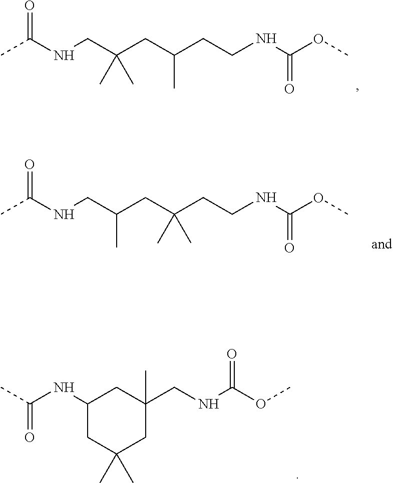

15. The free-radically polymerizable composition of claim 1, comprising
A) in an amount of from 10% to 60% by weight,
B) in an amount of from 30% to 85% by weight, and
C) in an amount of from 0.5% to 4% by weight,
based in each case on the total mass of the polymerizable composition.
